(19) United States
(12) Patent Application Publication
(10) Pub. No.: US 2004/0096896 A1
(43) Pub. Date: May 20, 2004

(54) Pattern recognition of serum proteins for the diagnosis or treatment of physiologic conditions

(75) Inventors: David B. Agus, Beverly Hills, CA (US); Mark D. Kvamme, Atherton, CA (US)

Correspondence Address: Pillsbury Winthrop LLP, Intellectual Property Group, Suite 2800, 725 South Figueroa Street, Los Angeles, CA, 90017-5406, US

(73) Assignee: CEDARS-SINAI MEDICAL CENTER

(21) Appl. No.: 10/294,270

(22) Filed: Nov. 14, 2002

(51) Int. Cl. G01N 33/53; G06F 19/00; G01N 33/48; G01N 33/50

(52) U.S. Cl. 435/7.1; 702/19

(57) ABSTRACT

Systems and methods of diagnosing and/or treating physiologic conditions based upon pattern recognition of serum protein profiles are provided. Mass spectrometry or other conventional techniques for creating a profile of serum proteins is employed, and a patient's profile is thereafter digitized for computational analysis. A pattern recognition algorithm is implemented to determine a degree of similarity between the patient's profile and other profiles stored in a database along with information describing the pathologic state of the individuals from whom such data was obtained. The degree of similarity may provide an indication of, for example, the way in which the patient may react to a particular clinical treatment or their predisposition to a particular disease condition. The methods and system of the present invention may be used to monitor the dynamic progression of disease pathology in a patient, and may be implemented via a computer network.

### FIELD OF THE INVENTION

Embodiments of the present invention are directed to pattern recognition of a serum protein profile obtained from a body fluid; particularly urine, blood, sweat, serum or plasma or a protein sample from a tumor. Methods and systems for diagnosing, prognosing and/or guiding treatment of physiological conditions based upon such pattern recognition of serum protein profiles are provided.

### BACKGROUND OF THE INVENTION

In any field of medicine, there is an ever-present interest in diagnosing a patient's condition as accurately as possible. There is a further interest in establishing the patient's prognosis and implementing the most effective therapeutic treatment or, treatments, especially in those instances where a variety of treatment options are available (including the option of not administering any therapy at all). It is often difficult to assess at the outset of treatment for a particular condition which therapeutic regimen will be most effective in a patient, and physicians may have to simply attempt treatment with a first therapy, and later implement a second therapy if that first therapy does not effect the desired physiologic response. Moreover, a patient's disease condition is dynamic in nature; it changes with time in response to treatment and as pathology progresses. This progression is generally monitored and treatment adjusted accordingly, yet it may be difficult to determine the most effective therapeutic treatment at various stages of clinical intervention.

Modern trends in medical research have highlighted the importance of understanding the genetic or other physiologic roots responsible for creating or facilitating the development of disease conditions. This research seeks to answer the basic question of why two people, similar in both intrinsic (e.g., age, weight, sex, height, body type, family history, other genetic factors, etc.) and extrinsic characteristics (e.g., environment, diet, stress level, etc.), can have widely divergent propensities to develop a particular disease condition. Or, why two such similar individuals that are afflicted with the same disease condition may have their condition respond entirely differently to a therapeutic treatment. There are likely a great number of factors that account for such differences, but even with our increased understanding of some of these factors, there is currently no quantitative tool with which to predict how a patient (or his condition) will respond to treatment. To the extent that some diagnostic and prognostic tools are available, there is none robust enough to account for both the intrinsic and extrinsic characteristics described above, as well as the wide array of pathological conditions that may confront a patient and the manner in which those conditions are likely to change with time and treatment.

With a profile of an individual's serum proteins, one may obtain something akin to a physiologic fingerprint; a quantitative, mathematical representation of the current state of that individual's internal biology. It may be indicative of the current progression of disease pathology, the nature of response to therapeutic intervention, or even the propensity with which one is predisposed to a particular illness. Such a profile may be obtained with mass spectrometry or similar techniques known to those of skill in the medical arts to characterize the proteins present in a particular sample. However, without a means by which to interpret this data on a large scale such as by comparing it to similar data obtained from others, the diagnostic potential of this data is left largely untapped. More specifically, the information contained in the data may not be understood without a benchmark against which to compare it; deviance or similarity to such a benchmark are likely to uncover untold volumes of information. More importantly, such information may have substantial implications if employed as a component of a diagnostic or prognostic method.

Therefore, there is a need in the art for a tool with which to access the information contained in the serum protein profile. Such a tool may have profound implications for the ways in which medicine is practiced. By way of example, a physician may be able to predict with unparalleled accuracy: the manifestation of a disease condition in a patient prior to that point in time where a conventional diagnostic test may screen for that condition; the reaction of a patient to a particular therapeutic treatment modality without the need to administer the treatment and to “see how it goes;” or the duration of and/or physiochemical changes associated with a disease condition that may differ among patients (e.g., severity of reaction to chemotherapy, potential for loss of sight with worsening of diabetes).

### SUMMARY OF THE INVENTION

In various embodiments, the present invention provides a diagnostic tool that implements pattern recognition analysis of a serum protein profile obtained from mass spectrometry or other techniques that can generate similar profiles. The serum protein profile may be based upon proteins obtained from body fluids such as urine, blood, sweat, plasma or serum, or from a protein sample from a tumor (obtained from fresh, frozen, or paraffin embedded tumor materials). It may be digitized and thereafter used to populate a database. In addition to the serum protein profile, patient clinical information may be input to the database to give supplementary physiologic meaning to the raw data. For example, a serum protein profile may be added to the database, along with information regarding an individual's disease condition, reaction/response to treatment, physiologic characteristics, and any other suitable or useful information. In one embodiment of the invention, serum samples from clinical trials databases with known outcomes of patients (e.g., therapeutic responses, side effect profile to therapeutics) may be analyzed to populate the database with protein profiles representing the outcome established in the clinical trial.

Once a database is generated, a patient's serum protein profile may be sampled by mass spectrometry or other conventional methodologies, and digitized in preparation for analysis with a pattern recognition algorithm or similar computational application. The patient's digitized profile may be iteratively compared with the serum protein profiles and associated clinical data included in the database, to identify pattern similarities therewith or differences therefrom. In this manner, one may, for instance, identify a level of similarity between a sampled serum profile from a patient with Alzheimer's Disease and a database profile or set of database profiles of individuals that also had Alzheimer's Disease and that responded positively to treatment with, e.g., ARICEPT (donepezil HCl; available from Pfizer, Inc.) or EXELON (rivastigmine tartrate; available from Novartis Pharmaceutical Corporation). Such individuals may be identified through involvement with a clinical trial of the therapeutic or physician-reported outcome outside of a clinical trial. One may therefore have a quantitative diagnostic tool with which to predict a likelihood of success with ARICEPT or EXELON for the patient, based upon pattern similarities with the serum protein profiles from individuals that responded positively to such treatment. Conversely, a notable difference between the patterns of individuals that responded positively to such treatment when compared with the patient's profile may translate to a low probability of success with these treatments for the patient. This is simply one example; the system and methods of the present invention may be extended to any physiologic condition or disease state.

The diagnostic/prognostic tool of the present invention may further be used to monitor the dynamic progression of a patient's medical condition. As a patient reacts and/or responds to clinical intervention, the propriety of various treatment alternatives may change. For instance, whereas kinase inhibitor therapy may have been a promising treatment in the early stages of prostate cancer, perhaps a resistance has manifested in a patient with time. By sampling the patient's serum profile again at a later stage of disease pathology and after administration of kinase inhibitor therapy;, a physician may then seek out alternative treatments by looking for similarities between the patient's updated serum profile and the profiles of others that arrived at a similar point in disease pathology.

In yet another aspect of the present invention, the database or databases created in accordance with the present invention may be accessible via a computer network, thereby enabling physicians in remote locations to access a centralized repository of information. Centralizing data such as this may speedily create a vast library of serum profiles and associated data (e.g., from various clinical studies) that may be used by countless numbers of physicians to provide higher quality care to their patients. Moreover, since each new serum profile that is analyzed with the database is potentially a new data set with which to populate the database, the database may grow exponentially as more researchers and physicians have access to the same.

### BRIEF DESCRIPTION OF THE DRAWINGS

FIG. 1 depicts a flow chart representation of pattern recognition of a serum protein profile in accordance with an embodiment of the present invention.

FIG. 2 depicts a system for pattern recognition of a serum protein profile including a database populated with serum protein profiles and associated clinical information. The device is illustratively depicted configured on a computer network.

FIG. 3 depicts a digitized serum protein profile in accordance with an embodiment of the present invention.

FIG. 4 illustrates a database architecture according to an embodiment of the present invention.

FIG. 5 illustrates a flow chart diagram of patient pattern logic according to an embodiment of the present invention.

FIG. 6 illustrates a flow chart diagram of mass spectrometry pattern logic according to an embodiment of the present invention.

FIG. 7 illustrates rank table combinations to generate a final table according to an embodiment of the present invention.

### DETAILED DESCRIPTION OF THE INVENTION

The present invention is based on a combination of a technique used in medicine to create a profile of serum proteins, with pattern recognition analysis generally employed in computing systems; thereby creating a tool for diagnosing and/or treating medical and other physiologic conditions. Techniques used to create a profile of serum proteins generally involve sampling a body fluid from an individual, and analyzing the serum proteins contained therein. The results of such an analysis may be embodied in a profile (i.e., a series of data points) of the serum proteins contained in the body fluid, which may be indicative of a particular medical or physiologic state in that individual. In accordance with various embodiments of the present invention, this “test” profile may be digitized (or originally created in digital format), and thereafter examined by pattern recognition analysis for a degree of similarity with another profile or set of other profiles stored in a database. The profile(s) in the database may describe a physiologic or other medical condition, and the degree of similarity between the test profile and the profile(s) stored in the database may thus indicate a likelihood of the individual having or developing the physiologic or other medical condition described by the profile(s) in the database.

Various body fluids may be extracted from an individual and examined to generate a test profile in accordance with various embodiments of the present invention. Such body fluids may include, but are in no way limited to, blood (including whole blood as well as its plasma and serum), urine, sweat, pulmonary secretions, tears, and a protein sample from a tumor (obtained from fresh, frozen, or paraffin embedded tumor materials) (each of which is hereinafter included in the term “serum”). In a preferred embodiment, one extracts and examines a sample of blood serum from a mammal. Body fluids obtained during the course of clinical trials may be particularly advantageous for use in accordance with various embodiments of the present invention, especially to populate the database described in further detail in the ensuing discussion.

Once extracted, any conventional technique may be used to generate a serum protein profile in accordance with various embodiments of the instant invention, as will be readily appreciated by one of skill in the art. Examples of such conventional techniques may include, but are in no way limited to, mass spectrometry, high pressure liquid chromatography (HPLC), and two-dimensional gel electrophoresis, or other mechanisms of demonstrating a multi-dimensional representation of the pattern of proteins in an individual's body fluid. In a preferred embodiment, mass spectrometry is utilized.

Mass spectrometry analysis may account for a variety of characteristics of the proteins sought to be profiled, including, but in no way limited to, molecular size, charge, and other characteristics well known to those of skill in the art of mass spectrometry. For instance, a size exclusion process may be implemented to limit the analysis to smaller serum proteins and protein fragments (e.g., degraded proteins). Appropriate testing parameters for these characteristics may be readily determined by routine experimentation by one possessing such skill. Preferably, only small proteins (e.g., proteins no larger than approximately 50 kD, and most preferably no larger than approximately 20 kD) are examined by mass spectrometry. Other mechanisms to enhance the patient sample for protein analysis may include mechanisms that-eliminate albumin, immunoglobulin and/or other “dominant” proteins that may mask or obfuscate the proper detection of other proteins in such an analysis.

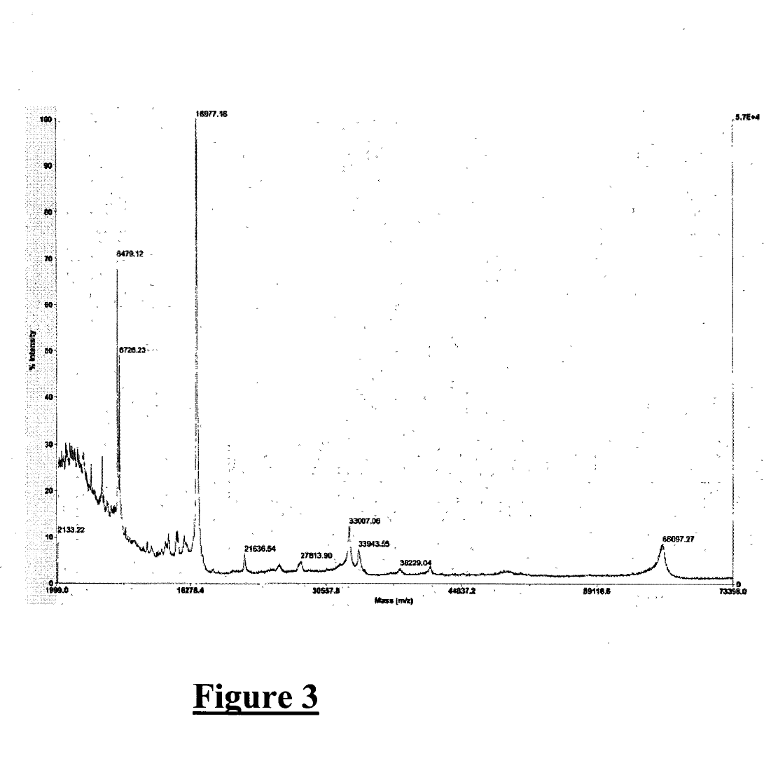

A serum protein profile may be generated directly into a digital readout by the equipment utilized to create the profile. In those embodiments where a profile is output in a manner other than a digital output, however, any suitable, conventional technique may be used to digitize the profile. By way of example, the readout from the protein profile may be output as an American Standard Code for Information Interchange (ASCII) file. An example of a digitized serum protein profile is depicted in FIG. 3.

Pattern recognition software is commercially available and conventionally implemented for a variety of purposes, such as electronic voice recognition, computer virus detection, and the like. By way of example, U.S. Pat. Nos. 6,154,773 and 6,304,523 each describe a fuzzy comparison algorithm suitable for determining whether two audio compact discs include approximately the same content. Any suitable pattern recognition/comparison scheme for data and/or images may be utilized.

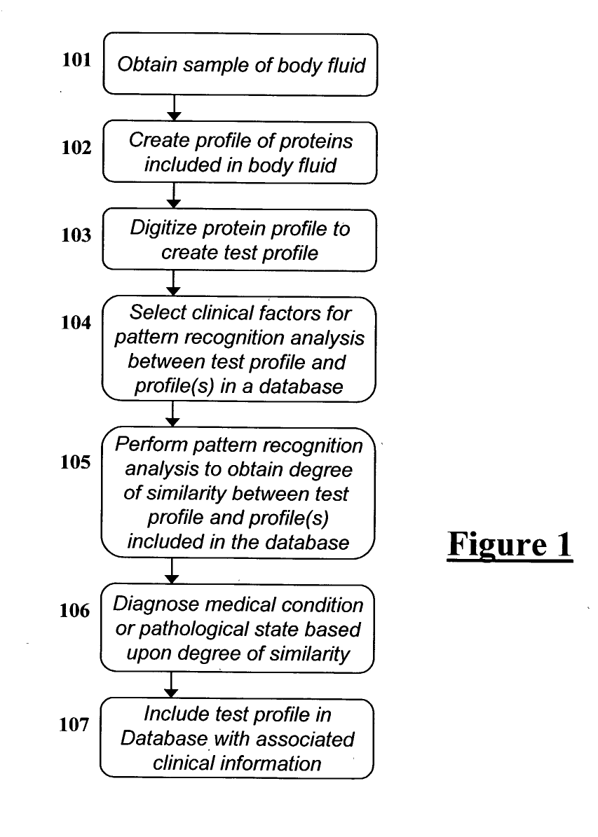

As depicted in FIG. 1, one may obtain a sample of body fluid 101 from an individual and create a profile of proteins included in the body fluid 102 by any suitable mechanism. If this information is not already in a digital format, then one may digitize the protein profile to create a test profile 103; although this may not be necessary in those embodiments wherein the equipment used to generate the profile of proteins outputs the serum protein profile in digital form. One may select clinical factors for pattern recognition analysis between the test profile and profiles stored in a database 104, such as, by way of example, the medical condition (e.g., prostate cancer), the efficacy of a particular treatment regime (e.g., “TAXOL was ineffective” (TAXOL: paclitaxel; available from Bristol-Myers Squibb Oncology/Immunology Division)), the point in the progression of the disease state at which the data was obtained (e.g., “immediately following radical prostatectomy”), and any number of other clinical factors. The selected clinical factors may be utilized to target the comparative, pattern recognition analysis to search, either exclusively or primarily, those entries in the database that may have relevance for the particular condition sought to be treated and/or identified. Pattern recognition analysis may then be performed to obtain a degree of similarity between the test profile and other protein profiles in the database 105. Based upon the degree of similarity (or dissimilarity), a medical condition or pathological state may be diagnosed 106, and the test profile along with its associated clinical data may thereafter be included in the database 107.

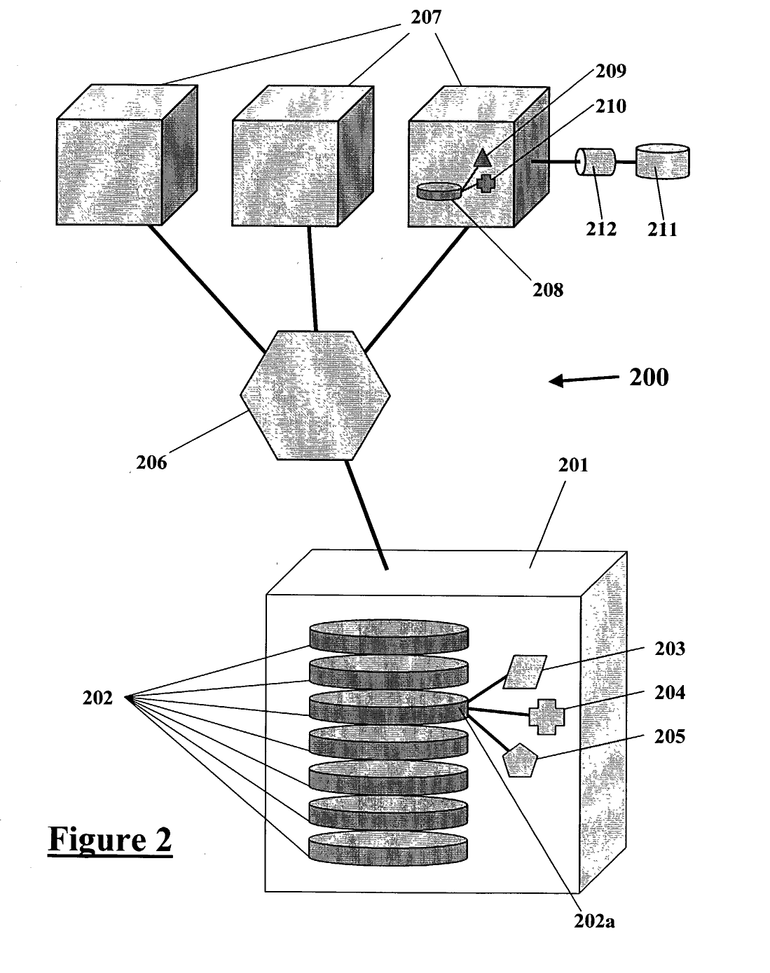

FIG. 2 depicts a system 200 for pattern recognition of a serum protein profile in accordance with an embodiment of the present invention. The system 200 may include a database 201 populated with serum protein profiles 202, each further including its individual, associated clinical information. Clinical information may include the identity of a disease state, a treatment regime that was implemented for the treatment of that disease state, the efficacy of the treatment regime, the physical traits of the individual from whom the serum protein profile 202 was extracted, and the like. By:way of example, one serum protein profile 202a included in the database 201 is illustratively depicted with three fields of clinical information 203, 204, 205; however, any suitable number of fields of clinical information may be included for each serum protein profile 202 in the database 201. Moreover, the various serum protein profiles 202 stored in the database 201 may each have different types and amounts of clinical information available to describe them, and, as such, the number of fields of clinical information associated with each serum protein profile 202 may be different. For example, a large amount of information may be available for serum protein profiles 202 sampled from an individual participating in a clinical study, owing to the detailed nature of data collection generally associated with clinical studies and the validated clinical outcome. However, less detail may be available for serum protein profiles 202 obtained by other means than from a clinical trial patient.

The system 200 may further be adapted for configuration on a network 206, such as an intranet (e.g., a local area network) (e.g., for accessing the database 201 within a hospital or system of hospitals), or the Internet (e.g., to provide access to the database 201 by remote users unaffiliated with the owner of the database 201). Remote terminals 207 may thereby access the database 201. In various embodiments, remote users may subscribe or otherwise pay for such access to the database 201 and/or for permission to utilize pattern recognition analysis to obtain a degree of similarity between a test serum protein profile 208 with the serum protein profiles 202 populating the database 201. The test serum protein profile 208 may have particular clinical information associated therewith, and the amount and type of this information that is available may differ among test profiles 208, as described above with respect to the serum protein profiles 202 that populate the database 201. By way of example, the test profile 208 included in the remote terminal 207 is illustratively depicted with two fields of clinical information 209, 210. The type of clinical information associated with the test profile 208 may be either the same (e.g., 204 and 210) or different (e.g., 209) from the clinical information associated with serum protein profiles 202 populating the database 201.

A protein profile generating apparatus 211 may be included in the system 200 to create the test serum protein profile 208. The protein profile generating apparatus 211 is preferably a mass spectrometer or other analytic mechanism capable of generating a multi-dimensional representation of the pattern of proteins in an individual's body fluid. The test serum protein profile 208 may be digitized by a digitizing apparatus 212 in those instances where the mass spectrometer or other analytic mechanism 212 does not output the test serum protein profile 208 in a digital format. According to an embodiment of the-present invention, the clinical information, or patient data, and the mass spectrometry data may be stored together, or attached to each other.

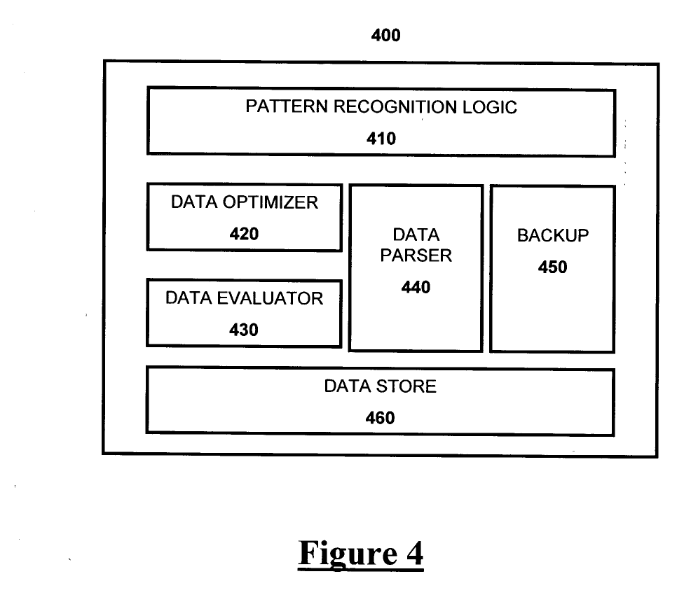

FIG. 4 illustrates a database architecture according to one embodiment of the present invention. The database 400 may include a pattern recognition logic module 410 having instructions to conduct patient data pattern comparisons and mass spectrometry data comparisons (further discussed below). The patient data and the mass spectrometry data may be stored in a machine-readable storage medium such as a data store 460 (e.g., a hard disk drive, an optical disc storage system, etc.). The patient data and the mass spectrometry data may be stored together (e.g., attached or linked), or stored separately within the data store 460, or other suitable storage medium. A data evaluator 430 determines the data to be extracted from the data store 460, and a data optimizer 420 ensures that the data is presented in an appropriate format and structure for processing. A data parser 440 examines and parses the relevant data to be processed and compared by the pattern recognition logic module 410. A backup module 450 may be included to provide redundant storage of data. However, other suitable database architectures than the embodiment illustrated in FIG. 4 may be utilized as well.

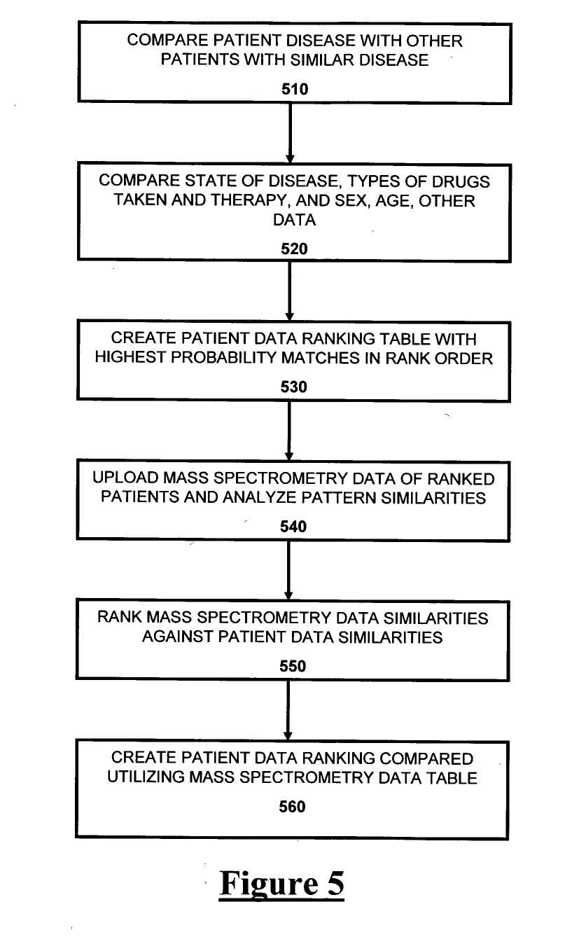

FIG. 5 illustrates a flow chart diagram of patient pattern logic according to an embodiment of the present invention. Raw data involving a particular patient is collected during a clinical trial, in the course of a routine physical examination with a treating physician, or at another appropriate time, for example, and entered into the database. The data may be converted into an extended markup language (XML) format, for example, for storage in the database. Once the raw patient data has been collected, the data is preferably stored in a central database. Based on the data obtained, usually from clinical trials, a particular patient's disease or symptom is compared 510 with that of all other patients' data stored in the database having a similar disease or symptom. Characteristics that may be compared 520, for example, include the state of the disease, the form of clinical intervention administered to date, and the sex, age, and other data.

For example, keyword-type comparisons of the data may be utilized to compare data from a particular patient with those of all other patients stored in the database having a, similar disease or symptom. For instance, if the particular patient of interest is a female having breast cancer, then an initial comparison may be made of patient data in the database of all female patients having breast cancer. And then, the comparison criteria may become narrower, such as all female patients over 65 years old having breast cancer. Moreover, the comparison criteria may become even more narrow, such as all female patients over 65 years old having breast cancer undergoing treatment with TAXOL. Depending on the specific characteristics of each patient in question and the number of patients in the database, the comparison criteria may be more detailed and narrow, or it may be more broad and general. Accordingly, keyword-type matches may be utilized in one embodiment of the present invention, for matches of the terms “female”, “65 years old”, “breast cancer”, “TAXOL”, etc., in the patient data. However, any suitable types of matching schemes may be utilized as well other than keyword-type matches.

Once the comparison of the patient data with all other patient data having the similar disease or symptom is conducted, a patient data ranking table of such comparison is created 530 with the highest probability matches in rank order. That is, a ranking of all other patients in the database having the highest probability of relevance with the patient in question based on the patient data comparison is performed. A threshold may be established as a cut-off of the ranking list, e.g., only those patients having better than 90% probability are listed in the patient ranking table.

The mass spectrometry data corresponding to each patient in the patient data ranking table is uploaded and analyzed 540 for pattern similarities. As mentioned above, for example, the mass spectrometry data may be in the XML format. Similarly to the creation of the patient data ranking table, the mass spectrometry data corresponding to each patient in the patient data ranking table is analyzed for similarities with the mass spectrometry data of the patient in question, and a ranking 550 of the highest probability of relevance to the mass spectrometry data of the patient in question is performed. A patient data ranking compared utilizing mass spectrometry data table is created 560 based on the highest probability of relevance matches of the mass spectrometry data of those patients in the patient ranking table in rank order to the mass spectrometry data of the patient in question.

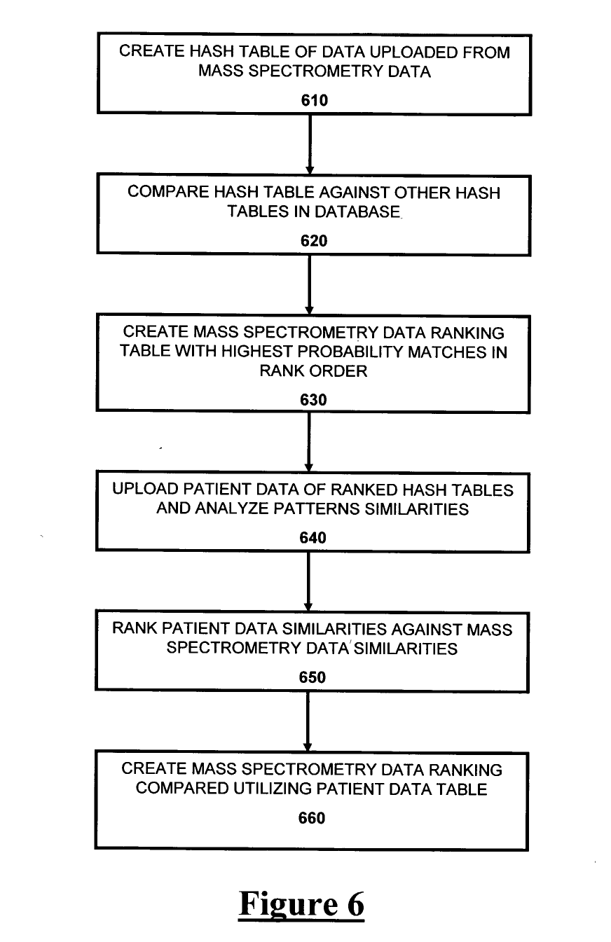

FIG. 6 illustrates a flow chart diagram of mass spectrometry pattern logic according to an embodiment of the present invention. A mass spectrometry data ranking table is created, similar to the ranking tables generated in FIG. 5. The mass spectrometry data ranking table is a ranking of the highest probability of relevance matches of the mass spectrometry data of all other patients in the database to the mass spectrometry data of the patient in question in rank order. According to an embodiment of the present invention, rather than comparing the raw mass spectrometry data to each other (which may be in the form of a graphics image or chart), a hash table may be created 610 from the mass spectrometry data for each patient in the database. For example, a hash table may include the values of —1, 0, and +1, for each field within the mass spectrometry data, and the hash table provides a simplified table for which comparisons may be more efficiently made. That is, in this one embodiment, the comparisons may be made by matches in each field of the hash table having only three possible values. The hash tables may also be utilized in the comparisons of the mass spectrometry data in FIG. 5. Accordingly, once the hash tables of the mass spectrometry data-for all of the patients in the database have been created, they are compared 620 with the hash table of the patient in question for pattern similarities.

A mass spectrometry data ranking table is created 630 based on the comparisons of the hash table of the patient in question with the hash tables of all other patients in the database. The mass spectrometry data ranking table includes a list of the highest probability matches in rank order of the hash tables of all other patients in the database compared to the hash table of the patient in question. That is, a ranking of the hash tables of all other patients in the database having the highest probability of relevance with the hash table of the patient in question is performed. A threshold, too, may be established as a cut-off of the ranking list (e.g., only those patients having better than 90% probability are listed in the mass spectrometry data ranking table).

The patient data corresponding to each patient in the mass spectrometry data ranking table is uploaded and analyzed 640 for pattern similarities. Similarly to the comparison of the patient data above in FIG. 5, the patient data corresponding to each patient in the mass spectrometry data ranking table is analyzed for similarities with the patient data of the patient in question, and a ranking 650 of the highest probability of relevance of the patient data of those patients on the mass spectrometry data ranking table with the patient data of the patient in question is performed. A mass spectrometry data ranking compared utilizing patient data table is created 660 based on the highest probability of relevance matches of the patient data of those patients in the mass spectrometry data ranking table in rank order to the patient data of the patient in question.

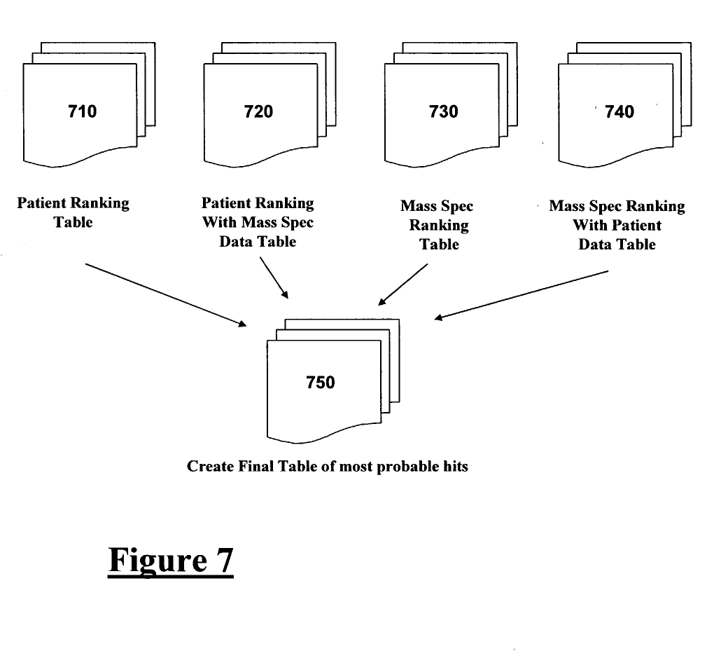

FIG. 7 illustrates rank table combinations to generate a final table according to an embodiment of the present invention. Following the completion of the comparisons in FIGS. 5 and 6, four tables are created: (1) a patient data ranking table 710 (see FIG. 5); (2) a patient data ranking compared utilizing mass spectrometry data table 720 (see FIG. 5) (i.e., a table of the highest probability of relevance matches in rank order of the mass spectrometry data of the patients in the patent data ranking table 710); (3) a mass spectrometry data ranking table 730 (see FIG. 6); and (4) a mass spectrometry data ranking compared utilizing patient data table 740 (see FIG. 6) (i.e., a table of the highest probability of relevance matches in rank order of the patient data of the patients in the mass spectrometry data ranking table 730). Utilizing each of these four tables 710, 720, 730, 740, a final table 750 is generated of a ranking of the highest overall probability of relevance of all other patients in the database compared to the patient in question. Based on the final table 750, which may be forwarded to a physician for review, it is determined with a certain probability (of which the threshold may also be set for listing in the final table 750), likely outcomes of treatment, reactions to drug usage, progression of the disease, etc., of the patient in question based on the clinical trial data (or data obtained by other means) of other patients in the database having high rankings in the final table 750 generated by the pattern recognition system according to embodiments of the present invention. For example, by reviewing the clinical trial outcome for a patient in the database that has an overall high probability match in the final table 750 to that of the patient in question, the clinical trial data of the high probability matching patient may be utilized to predict likely courses of treatment, or a likely progression of a disease utilizing a particular course of treatment or medication as implemented with the high probability matching patient.

### EXAMPLES

The following examples are typical of the procedures that may be used to treat or diagnose physiologic conditions, such as by predicting the efficacy of therapeutic treatment strategies which may be used to treat such conditions, and to predict the progression of disease pathology in accordance with various embodiments of the present invention. Modifications of these examples will be readily apparent to those skilled in the art who seek to implement the methods and system of the present invention in a manner that differs from that described herein.

### Example 1

### Treating a Physiologic Condition by Pattern Recognition Analysis of a Test Serum Protein Profile

Blood is sampled from a prostate cancer patient who recently underwent a radical prostatectomy. A serum protein profile (“test profile”) is generated by a two-dimensional readout from mass spectrometry of proteins and protein fragments less than 20 kD in size in the blood serum. The test profile is digitized and loaded into pattern recognition software residing in a computer terminal, along with the following information: (1) the patient has prostate cancer; (2) the patient recently underwent a complete prostatectomy; (3) the patient weighs 174 pounds; (4) the Gleason score of the pathologic sample removed from the patient was 4+3 (standard pathologic grading done routinely by pathologists who review the patient's disease tissue); (5) the patient is age 64 years; (6) the serum prostate-specific antigen (PSA) of the patient prior to surgery was 9.7 ng/ml; and (7) the patient's tumor was felt to be a Stage 2a clinically prior to surgery.

The computer terminal is connected via an electronic communications network to a database populated with serum protein profiles generated from individuals participating in clinical studies of various cancer therapies. The serum protein profiles were originally obtained from individuals with different types of cancer at different stages in their cancer's pathological progression. The individuals received different forms of clinical intervention with varying degrees of success, as is generally the case with clinical studies of this nature. For example, in the case mentioned above, a database from a patient population who underwent radical prostatectomy with known outcome (e.g., cure, local recurrence, distant metastatic recurrence and the time kinetics of the outcome) will have been analyzed previously by the methodology described previously. Thus, the patient's (in this example, the patient mentioned above) protein profile is compared with the samples from the validated clinical database, and the protein pattern similarity is correlated to outcome and disease phenotype. The ultimate readout is a statistical description of the likelihood of various clinical outcomes for the patient, based on the outcomes of the patient samples (and their respective outcomes) already in the database.

The pattern recognition analysis is performed to find mathematically significant consistencies between the test profile and a profile or profiles contained in the database. By virtue of the additional clinical information supplied with the test profile, the pattern recognition analysis may be limited to those serum protein profiles in the database that were obtained from individuals treated for prostate cancer that underwent a complete prostatectomy. The other clinical information (i.e., weight, age, Gleason's score, serum PSA value, etc.) may be utilized to further narrow the search and comparison parameters of the analysis, and may provide yet further insight for the physician. For instance, there may be a marked difference in the efficacy of various medications among prostate cancer patients based on their age. Searches may therefore be limited or generalized based upon the information input to the system, and a degree of similarity is thereafter generated to a profile or set of profiles in the database.

In this instance, an 86% degree of similarity (generated by standard biostatistical information) is generated for the test profile with a set of profiles in the database from individuals who had prostate cancer and underwent a complete prostatectomy, had a recurrence and additionally responded positively to treatment with TAXOL. The physician therefore determines that TAXOL may be an appropriate treatment at this stage of clinical intervention for continued treatment for the patient. More specifically, the physician bases this determination on there being an approximately 86% chance of success with TAXOL owing to the similarities between his patient and the set of profiles in the database.

### Example 2

### Dynamically Treating a Physiologic Condition by Pattern Recognition Analysis of a Test Serum Protein Profile

A test profile is generated by mass spectrometry of proteins and protein fragments less than 20 kD in size in the blood serum from the patient described in Example 1, above, six months after the initial pattern recognition and immediate initiation of treatment with TAXOL. Another pattern recognition analysis now performed with the same database, and the associated clinical information is amended to include the six month period of treatment with TAXOL.

At this point, there is now a 37% degree of statistical similarity generated for the test profile with a set of profiles in the database from individuals who had prostate cancer, underwent a complete prostatectomy, had a recurrence and additionally responded positively to-treatment with TAXOL. The patient also has a 69% statistical degree of similarity with a similar patient subset which had been known to be refractory to TAXOL. The treating physician therefore determines that TAXOL may no longer be an appropriate therapeutic treatment for this patient.

### Example 3

### Predicting the Progression of Disease Pathology by Pattern Recognition Analysis of a Test Serum Protein Profile

Blood is sampled from a man with human immunodeficiency virus (HIV), after seroconversion of the virus but while he remains asymptomatic. He is currently being treated with a “cocktail” of VIRACEPT (nelfinavir mesylate; available from Agouron Pharmaceuticals, Inc.), RETROVIR (zidovudine (AZT); available from Glaxo SmithKline), and VIDEX (didanosine (ddl); available from Bristol-Myers Squibb Company). A test profile is generated by mass spectrometry of proteins and protein fragments less than 20 kD in size in his blood serum. The test profile is digitized and loaded into pattern recognition software residing in a computer terminal, along with the following information: (1) the individual has HIV; (2) he is at a stage in HIV progression of post seroconversion yet asymptomatic; and (3) he is presently on a treatment regime consisting of VIRACEPT, RETROVIR, and VIDEX.

The computer terminal is connected via an intranet to a database populated with serum protein profiles generated from individuals participating in clinical studies of various therapies for HIV and acquired immune deficiency syndrome (AIDS), as well as serum protein profiles obtained from individuals previously examined by the physician seeking treatment information for this patient. The individuals were at various stages of HIV/AIDS, and received different forms of clinical intervention with varying degrees of success.

The pattern recognition analysis is performed to find mathematically significant consistencies, by biostatistical analysis, between the test profile and a profile or profiles contained in the database. By virtue of the additional clinical information supplied with the test profile, the pattern recognition analysis is limited to those serum protein profiles in the database that were obtained from individuals who were HIV positive, were asymptomatic yet post seroconversion, and who were receiving cocktails consisting of a protease inhibitor (e.g., VIRACEPT) and at least one nucleoside reverse transcriptase inhibitor (e.g., RETROVIR, VIDEX).

A 74% degree of similarity is generated for the test profile with a series of profiles in the database from individuals at a similar stage of disease pathology receiving a cocktail, each of whom remained free from opportunistic infection associated with full-blown AIDS for at least 9 years. The physician therefore concludes that this individual has a defined statistical likelihood to have a similar disease progression with the present treatment regime.

### Example 4

### Diagnosing Physiologic Conditions by Pattern Recognition Analysis of a Test Serum Protein Profile

Blood is sampled from a 64-year old woman exhibiting some short term memory loss, and both a demonstrated difficulty in telling time and in handling simple mathematic calculations. A test profile is generated by mass spectrometry of proteins and protein fragments less than 20 kD in size in her blood serum. The mass spectrometry equipment generates a digital test profile, which is loaded into pattern recognition software residing in a computer terminal, along with the following information: (1) the individual is a 64-year old woman; and (2) she exhibits short term memory loss and difficulty with numbers.

The computer terminal is connected via an intranet to a database populated with serum protein profiles generated from individuals diagnosed with a variety of neurodegenerative disorders, such as Alzheimer's Disease, Parkinson's Disease, and the like.

The pattern recognition analysis is performed to find mathematically significant consistencies between the test profile and a profile or profiles contained in the database. By virtue of the additional clinical information supplied with the test profile, the pattern recognition analysis is limited to those serum protein profiles in the database that were obtained from individuals who were female at or about the age of 64 exhibiting short term memory loss and difficulty with numbers.

A 24% degree of similarity is generated for the test profile with a series of profiles in the database from women in their early to mid-sixties with similar memory loss and difficulty with numbers that were diagnosed with Vascular Dementia, but a 92% degree of statistical similarity is generated with a series of profiles with similar associated clinical information, yet for individuals who were diagnosed with Alzheimer's Disease. The physician therefore concludes that this individual is statistically more likely to have Alzheimer's Disease than Vascular Dementia.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. A system for pattern recognition of a test profile, comprising:
a test profile of a patient's serum proteins;
a database including at least one serum protein profile; and
a pattern recognition algorithm to compare the test profile with the at least one serum protein profile included in the database.

2. The system of claim 1, wherein
the test profile is associated with clinical information to identify physiologic or medical data for the patient, and
the pattern recognition algorithm uses the clinical information to narrow a scope of an analysis performed with the pattern recognition algorithm.

3. The system of claim 1, wherein the database further comprises clinical information associated with each of the at least one serum protein profile to identify physiologic or medical data for the at least one serum protein profile.

4. The system of claim 1, further comprising a protein profile generating apparatus to generate the test profile, and selected from the group consisting of a mass spectrometer, a high performance liquid chromatography apparatus, and a two-dimensional gel electrophoresis apparatus.

5. The system of claim 1, further comprising a digitizing apparatus to translate the test profile into a digital format.

6. The system of claim 1, wherein the patient's serum proteins are sampled from a body fluid of the patient, the body fluid being selected from the group consisting of blood, whole blood, blood plasma, blood serum, urine, sweat, pulmonary secretions, tears, and a protein sample from a tumor.

7. The system of claim 1, wherein the patient's serum proteins are less than about 20 kD in size.

8. The system of claim 1, further comprising a network to provide electronic communication between the database and a remote computer terminal.

9. The system of claim 8, further comprising at least one remote computer terminal in electronic communication with the network, the remote computer terminal to compare the test profile with the at least one serum protein profile included in the database.

10. A method for treating a physiologic condition in a patient, comprising:
analyzing a test profile of serum proteins from the patient with a pattern recognition algorithm to compare the test profile to at least one serum protein profile included in a database; and
deciding on a course of treatment for the patient based upon a result of the pattern recognition algorithm.

11. The method of claim 10, wherein the database further comprises clinical information associated with each of the at least one serum protein profile to identify physiologic or medical data for the at least one serum protein profile.

12. The method of claim 11, further comprising:
including at least one clinical factor with the test profile to narrow a scope of an analysis performed with the pattern recognition algorithm, the at least one clinical factor identifying physiologic or medical data for the patient.

13. The method of claim 10, wherein the result of the pattern recognition algorithm is a degree of similarity between the test profile and at least one serum protein profile included in the database.

14. The method of claim 10, further comprising:
obtaining a sample of a body fluid from the patient, the body fluid further comprising serum proteins; and
creating the profile of serum proteins with a protein profile generating apparatus.

15. The method of claim 14, wherein the body fluid is selected from the group consisting of blood, whole blood, blood plasma, blood serum, urine, sweat, pulmonary secretions, tears, and a protein sample from a tumor, and the protein profile generating apparatus is selected from the group consisting of a mass spectrometer, a high performance liquid chromatography apparatus, and a two-dimensional gel electrophoresis apparatus.

16. The method of claim 10, wherein the serum proteins are less than about 20 kD in size.

17. The method of claim 10, further comprising:
digitizing the profile of serum proteins to translate the profile of serum, proteins into a digital format.

18. The method of claim 12, wherein after analyzing the test profile of serum proteins from the patient with the pattern recognition algorithm, the method further comprises:
including the test profile of serum proteins and at least one clinical factor in the database.

19. The method of claim 10, further comprising:
inputting the test profile of serum proteins into a computer terminal; and
accessing the database with the computer terminal via a network in electronic communication with the database.

20. A method for diagnosing a physiologic condition in a patient, comprising:
analyzing a test profile of serum proteins from the patient with a pattern recognition algorithm to compare the test profile to at least one serum protein profile included in a database; and
diagnosing a condition in the patient based upon a result of the pattern recognition algorithm.

21. The method of claim 20, wherein the database further comprises clinical information associated with each of the at least one serum protein profile to identify physiologic or medical data for the at least one serum protein profile.

22. The method of claim 21, further comprising:
including at least one clinical factor with the test profile to narrow a scope of an analysis performed with the pattern recognition algorithm, the at least one clinical factor identifying physiologic or medical data for the patient.

23. The method of claim 20, wherein the result of the pattern recognition algorithm is a degree of similarity between the test profile and at least one serum protein profile included in the database.

24. The method of claim 20, further comprising:
obtaining a sample of a body fluid from the patient, the body fluid further comprising serum proteins; and
creating the profile of serum proteins with a protein profile generating apparatus.

25. The method of claim 24, wherein the body fluid is selected from the group consisting of blood, whole blood, blood plasma, blood serum, urine, sweat, pulmonary secretions, tears, and a protein sample from a tumor, and the protein profile generating apparatus is selected from the group consisting of a mass spectrometer, a high performance, liquid chromatography apparatus, and a two-dimensional gel electrophoresis apparatus.

26. The method of claim 20, wherein the serum proteins are less than about 20 kD in size.

27. The method of claim 20, further comprising:
digitizing the profile of serum proteins to translate the profile of serum proteins into a digital format.

28. The method of claim 22, wherein after analyzing the test profile of serum proteins from the patient with the pattern recognition algorithm, the method further comprises:
including the test profile of serum proteins and at least one clinical factor in the database.

29. The method of claim 20, further comprising:
inputting the test profile of serum proteins into a computer terminal; and
accessing the database with the computer terminal via a network in electronic communication with the database.

30. A method of pattern recognition of serum proteins for diagnosis or treatment of physiological conditions, comprising:
generating a patient data ranking table;
generating a patient data ranking compared utilizing mass spectrometry data table;
generating a mass spectrometry data ranking table;
generating a mass spectrometry data ranking compared utilizing patient data table; and
generating a final table of highest overall probability of relevance matches based on the patient data ranking table, the patient data ranking compared utilizing mass spectrometry data table, the mass spectrometry data ranking table, and the mass spectrometry data ranking compared utilizing patient data table, wherein the final table is reviewed for diagnosis or treatment of a patient.

31. The method according to claim 30, wherein generating the patient data ranking table includes:
comparing patient data of the patient to patient data of other patients; and
ranking the patient data of the other patients based on highest probability of relevance to the patient data of the patient.

32. The method according to claim 31, wherein the patient data is at least one of a disease, a state of disease, types of drugs taken, types of therapies taken, a sex, and an age.

33. The method according to claim 30, wherein generating the patient data ranking compared utilizing mass spectrometry data table includes:
providing and analyzing mass spectrometry data of patients listed in the patient data ranking table; and
ranking the mass spectrometry data of the patients listed in the patient data ranking table based on highest probability of relevance to mass spectrometry data of the patient, wherein the mass spectrometry data is obtained from a mass spectrometry analysis of the serum proteins.

34. The method according to claim 33, wherein the mass spectrometry data of the patients listed in the patient data ranking table and the mass spectrometry data of the patient are in each in a hash table.

35. The method according to claim 30, wherein generating the mass spectrometry data ranking table includes:
comparing mass spectrometry data of the patient to mass spectrometry data of other patients; and
ranking the mass spectrometry data of the other patients based on highest probability of relevance to the mass spectrometry data of the patient, wherein the mass spectrometry data is obtained from a mass spectrometry analysis of the serum proteins.

36. The method according to claim 35, further including:
creating a hash table for each of the mass spectrometry data of the other patients and the mass spectrometry data of the patient; and
comparing the hash table of the patient to hash tables of the other patients.

37. The method according to claim 30, wherein generating the mass spectrometry data ranking compared utilizing patient data table includes:
providing and analyzing patient data of patients listed in the mass spectrometry data ranking table; and
ranking the patient data of the patients listed in the mass spectrometry data ranking table based on highest probability of relevance to patient data of the patient.

38. The method according to claim 37, wherein the patient data is at least one of a disease, a state of disease, types of drugs taken, types of therapies taken, a sex, and an age.

39. A program code storage device, comprising:
a machine-readable storage medium; and
machine-readable program code, stored on the machine-readable storage medium, having instructions to
generate a patient data ranking table,
generate a patient data ranking compared utilizing mass spectrometry data table,
generate a mass spectrometry data ranking table,
generate a mass spectrometry data ranking compared utilizing patient data table, and
generate a final table of highest overall probability of relevance matches based on the patient data ranking table, the patient data ranking compared utilizing mass spectrometry data table, the mass spectrometry data ranking table, and the mass spectrometry data ranking compared utilizing patient data table, wherein the final table is reviewed for diagnosis or treatment of a patient.

40. The program code storage device according to claim 39, wherein the instructions to generate the patient data ranking table further includes instructions to:
compare patient data of the patient to patient data of other patients; and
rank the patient data of the other patients based on highest probability of relevance to the patient data of the patient.

41. The program code storage device according to claim 40, wherein the patient data is at least one of a disease, a state of disease, types of drugs taken, types of therapies taken, a sex, and an age.

42. The program code storage device according to claim 39, wherein the instructions to generate the patient data ranking compared utilizing mass spectrometry data table further includes instructions to:
provide and analyze mass spectrometry data of patients listed in the patient data ranking table; and
rank the mass spectrometry data of the patients listed in the patient data ranking table based on highest probability of relevance to mass spectrometry data of the patient, wherein the mass spectrometry data is obtained from a mass spectrometry analysis of serum proteins.

43. The program code storage device according to claim 42, wherein the mass spectrometry data of the patients listed in the patient data ranking table and the mass spectrometry data of the patient are in each in a hash table.

44. The program code storage device according to claim 39, wherein the instructions to generate the mass spectrometry data ranking table further includes instructions to:
compare mass spectrometry data of the patient to mass spectrometry data of other patients; and
rank the mass spectrometry data of the other patients based on highest probability of relevance to the mass spectrometry data of the patient, wherein the mass spectrometry data is obtained from a mass spectrometry analysis of serum proteins.

45. The program code storage device according to claim 44, wherein the instructions to generate the mass spectrometry data ranking table further includes instructions to:
create a hash table for each of the mass spectrometry data of the other patients and the mass spectrometry data of the patient; and
compare the hash table of the patient to hash tables of the other patients.

46. The program code storage device according to claim 39, wherein the instructions to generate the mass spectrometry data ranking compared utilizing patient data table further includes instructions to:
provide and analyze patient data of patients listed in the mass spectrometry data ranking table; and
rank the patient data of the patients listed in the mass spectrometry data ranking table based on highest probability of relevance to patient data of the patient.

47. The program code storage device according to claim 46, wherein the patient data is at least one of a disease, a state of disease, types of drugs taken, types of therapies taken, a sex, and an age.
